(19) United States
(12) Patent Application Publication
(10) Pub. No.: US 2022/0187275 A1
(43) Pub. Date: Jun. 16, 2022

(54) SYSTEMS AND METHODS FOR DIRECTED FORMATION OF SIZE-CONTROLLED MULTI-CELLULAR STRUCTURES AND MEASUREMENT OF FORCES GENERATED BY THE SAME

(71) Applicant: Forcyte Biotechnologies, Inc., Los Angeles, CA (US)

(72) Inventors: Ivan Pushkarsky, Los Angeles, CA (US); Junyi Zhao, Torrance, CA (US)

(21) Appl. No.: 17/438,307

(22) PCT Filed: Mar. 12, 2020
(86) PCT No.: PCT/US20/22439

§ 371 Date: Sep. 10, 2021

Related U.S. Application Data
(60) Provisional application No. 62/817,305, filed on Mar. 12, 2019.

(51) Int. Cl. G01N 33/483

(52) U.S. Cl. CPC G01N 33/483; G01N 2203/0089

(57) ABSTRACT

An assay for measuring cell-generated forces, is disclosed. The assay includes a first substrate comprising an elastic material having a Young's modulus of between about 0.1 kPa to about 50 kPa and a geometric micropattern disposed onto a surface of the substrate. The micropattern is further comprised of at least one segment consisting of a fluorophore-conjugated material, a material with cell adhesion properties, and has an interior region.

### CROSS-REFERENCE TO RELATED APPLICATIONS

This application claims the benefit of U.S. Provisional Application No. 62/817,305, filed on Mar. 12, 2019, the disclosure of which is hereby incorporated by reference in its entirety.

### BACKGROUND

3-dimensional (3D) cellular tissuoids, organoids, spheroids, or cellular ensembles/structures are thought to be better physiological models for human health and disease when compared to strictly 2-dimensional (2D) cell cultures, owing to their 3D growth and interactions, complex architectural structure, and dynamic cell-cell interactions. As such, cellular tissuoids are used to assess the effects of drugs in discovery and pre-clinical stages, as well as in toxicology and organ development applications and studies. Tissuoids have been formed with a variety of cell types representing various organs and tissue including various cancer cell lines, fibroblasts, stem cells, cardiac cells, hepatocytes and others. Within these tissuoid structures, cells better resemble the native architecture and collective organization that is found in whole organ tissues, including tissues capable of generating mechanical stresses to perform key biological function. Since these mechanical forces can become disrupted in disease, there is a need to model tissue-generated forces in the lab in a cost-effective and robust manner to allow for screening of new treatments. Currently, tissue-level force modeling is done using ex vivo tissue sections or strips in very low-throughput, such that drug screening is not practical. Thus, there is a need for systems and methods to measure the collective mechanical force generation of tissue-like multi-cellular structures (MCS) in a high-throughput and highly parallelized manner to efficiently and cost effectively characterize the therapeutic and toxicological effects of compounds for drug discovery and/or diagnostics applications.

There are several conventional techniques for forming cellular tissuoids, such as dispersed cell aggregation tissuoid formation, matrix-supported tissuoid formation and microfluidic-supported tissuoid formation. However, as the heterogeneous metabolism, gene expression and function in MCSs can be influenced by the MCS size, there is a need to produce uniformly size tissuoids which is currently difficult and requires high initial concentrations of cells. Therefore, there is also a need for new methods for directing the formation of size-controlled tissuoids to help increase reproducibility of cell force measurements and potentially increase throughput.

### SUMMARY

At least certain embodiments disclosed herein are generally directed towards systems and methods for studying cell contractile forces. More specifically, the present disclosure includes, among other things, the recognition that there is a need for systems and methods which can measure contractile forces of three-dimensional multi-cellular structures adhering to a two-dimensional planar surface to characterize the therapeutic and toxicological effects of compounds in drug discovery and/or diagnostics applications.

In one aspect, an assay for measuring cell-generated forces, is disclosed. The assay includes a first substrate comprising an elastic material having a Young's modulus of between about 0.1 kPa to about 50 kPa and a geometric micropattern disposed onto a surface of the substrate. The micropattern is further comprised of at least one segment consisting of a fluorophore-conjugated material, a material with cell adhesion properties, and has an interior region.

In another aspect, an array for measuring cell-generated forces, is disclosed. The array includes a first substrate comprising an elastic material having a Young's modulus of between about 0.1 kPa to about 50 kPa and a plurality of geometric micropatterns disposed onto a surface of the substrate. Each of the plurality of micropatterns is comprised of at least one segment consisting of a fluorophore-conjugated material, a material with cell adhesion properties, and an interior region.

In still another aspect, a method for producing an assay to measure cell-generated forces, is disclosed. A geometric micropattern is deposited onto a planar first substrate having a Young's modulus of between about 0.1 kPa to about 50 kPa. The micropattern is comprised of at least one segment consisting of a fluorophore-conjugated material, a material with cell adhesion properties, and an interior region. A plurality of target cells is provided proximate to the micropattern in conditions that promote adhesion of the target cells to the micropattern. The target cells are incubated in media in conditions that promote cell growth and within the interior region.

In yet another aspect, a system for measuring cell-generated forces, is disclosed. The system includes an assay, a first light source, a detection module and a computing device.

The assay is comprised of a planar substrate comprising an elastic material having a Young's modulus of between about 0.1 kPa to about 50 kPa and a geometric micropattern disposed on a top surface of the substrate. The micropattern is comprised of at least one segment consisting of a fluorophore-conjugated material, a material with cell adhesion properties, and an interior region.

The first light source is capable of emitting light at a first pre-determined wavelength to excite the fluorophore-conjugated material. The detection module is operable to capture fluorescent light emitted by the micropattern. The computing device capable of receiving image data from the detection module to measure dimensional changes in the micropattern.

In still yet another aspect, a method for measuring cell-generated forces, is disclosed. An assay is provided. The assay includes a first planar substrate comprising an elastic material having a Young's modulus of between about 0.1 kPa to about 50 kPa, a geometric micropattern disposed onto a top surface of the first planar substrate and a plurality of target cells confined within the boundaries of the micropattern. The micropattern is comprised of at least one segment consisting of a fluorophore-conjugated material, a material with cell adhesion properties, and an interior region. The assay is illuminated with a light source set at a predetermined wavelength and a dimensional change of the micropattern is measured.

Additional aspects will be evident from the detailed description, which follows, as well as the claims appended hereto and the drawings.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 illustrates an array for measuring cell forces, in accordance with some embodiments of the disclosure.

FIG. 2 illustrates one example of a MCS-supporting micropattern that can be loaded with a plurality of target cells to form a MCS for measuring cell forces, in accordance with some embodiments of the disclosure.

FIG. 3 illustrates illustrative shapes and configurations for the MCS-supporting micropatterns that support MCS growth and enable force generation measurements of the MCS, in accordance with some embodiments of the disclosure.

FIG. 4 is a flowchart showing a method for producing an assay to measure cell-generated forces, in accordance with some embodiments of the disclosure.

FIG. 5 depicts the explicit microfabrication process used to produce arrays of MCS-supporting patterns 100, in accordance with various embodiments.

FIG. 6 is a schematic diagram of a system for measuring cell-generated forces, in accordance with various embodiments.

FIG. 7 is a flowchart showing a method for measuring cell-generated forces, in accordance with various embodiments.

FIG. 8 illustrate fluorescent images of viable and contracting MCSs adhered to a MCS-supporting micropatterns, an of MCS that have been exposed to triton-X solution.

FIG. 9 illustrates MCS-supporting micropatterns that are contracted by adhered MCS before and after stimulation with histamine, as well as time-lapse data of kinetic micropattern contraction by MCS treated with histamine and formoterol.

FIG. 10 illustrates MCS-supporting micropatterns that are contracted by adhered MCS comprising cardiac myocytes and the changes of their respective areas as a result of contractions generated by the MCS onto the MCS-supporting micropatterns over a period of observation.

FIG. 11 illustrates additional MCS-supporting micropattern geometries that have been designed and experimentally used. It further depicts MCS-supporting micropatterns that are contracted by adhered MCS comprised of cardiac myocytes, conveying a relaxed and maximally contracted state for multiple instances of the MCS-supporting micropatterns, and presents a trace of this contraction over a period of observation.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

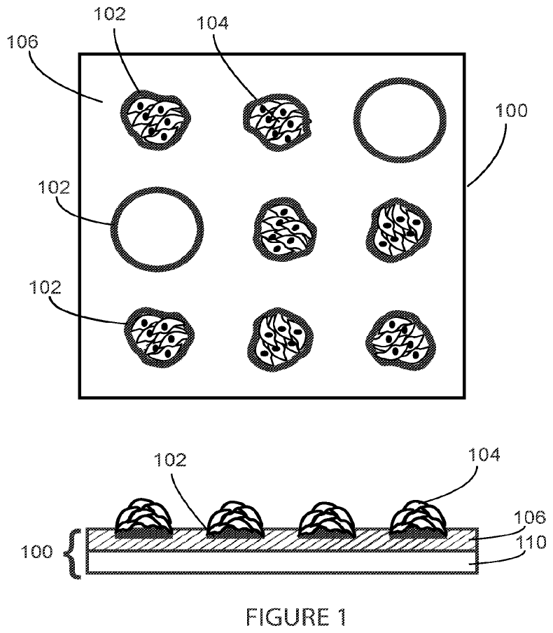

FIG. 1 illustrates an array 100 for measuring cell forces, in accordance with some embodiments of the disclosure. As depicted herein, the array 100 includes a plurality of multi-cellular structure (MCS)-supporting micropatterns 102 disposed onto or embedded within a first substrate 106. In various embodiments, the array 100 is comprised of three distinct layers, a first substrate 106 containing an array of MCS-supporting micropatterns is attached or disposed onto a rigid second substrate 110. The rigid second substrate provides an underlying supporting material that maintains planarity of the substrate, enabling simple and non-destructive handling of the substrate by humans and robots, and is optically transparent to enable imaging and transmission of light through the rigid second substrate. Adhesive forces inherent to the soft material comprising the first layer 106 enable robust adhesion to the rigid layer 110.

In various embodiments, the first substrate 106 is a planar elastic material that is comprised of polydimethylsiloxane (PDMS) or silicone elastomers. In other embodiments, the soft substrate 106 is a planar elastic material that is comprised of polyacrylamide hydrogels. In various embodiments, the first substrate 106 has a Young's modulus of between about 0.1 kilopascal (kPa) to about 50 kPa and is between about 10 and 300 microns thick.

In various embodiments, the second substrate 110 is comprised of a rigid or semi-rigid material that is, preferably, optically transparent. Examples, of rigid materials can include, but not limited to: glass, rigid plastics, crystalline materials, etc. This second substrate is secured to the soft substrate 106 opposite the side of the embedded MCS-supporting patterns 102 to provide rigid planar base for the soft substrate 106 to enable handling by humans and robots.

In various embodiments, the patterns 102 are disposed onto or embedded into a surface of the first substrate 106 and comprise a fluorophore-conjugated material with cell adhesion properties, or a plurality of materials of which at least one is adhesive to cells and at least one is fluorescent. That is, one of these materials, which may optionally include the fluorophore-conjugated material, includes components or elements that have a binding affinity to cells that come into contact with the material and the interior region of the MCS-supporting micropatterns is non-adhesive because it lacks adhesive fluorescent material and because it is blocked with Pluronic F-127. Examples of adhesive and fluorescent materials that can be used, include, but are not limited to fibronectin, fibrinogen, collagen type I, collagen type II, collagen type III, collagen type IV, gelatin, laminin, elastin, vitronectin, Matrigel, IgG, albumin, and poly-D-lysine, as well as other peptides and proteins. Any of these proteins may be conjugated to fluorescent moieties including red fluorescent protein (RFP), green fluorescent protein (GFP), Alexa Fluor 488, Alexa Fluor 555, Alexa Fluor 546, Alexa Fluor 568, Alex Fluor 647, Oregon Green, Fluorescein Isothiocyanate, tetramethyl rhodamine isothiocyanate, and other fluorescent molecules. In various embodiments, the conjugation chemistry between the fluorescent moieties and the adhesive molecules may involve amine-based and thiol-based conjugations.

Within each of the plurality of MCS-supporting micropatterns 102 is an interior region that is non-adhesive over which a multi-cellular structure (MCS) 104 comprised of a plurality of target cells spreads across without forming adhesions to this interior region. Cells, seeded in approximately quantities of 2 or more per MCS-supporting micropatterns, typically dispensed as between about 500 to 2500 cells per well on a 384-well plate, initially adhere to the segments comprising fluorescent and adhesive materials. Then, the substrate and the cells are incubated at 37 degrees Celsius so that they divide and grow, for at least one day and up to several weeks, in a controlled manner within the boundaries of the micropattern 102 and spread across without adhering to the interior region of the MCS-supporting micropattern 102, bridging the cell-adhesive segments that lie on the convex envelope of the MCS-supporting micropattern 102, and ultimately forming the MCS 104.

After the MCS 104 is formed, it can be tested with various chemical or biological agents to characterize their respective toxicological and/or therapeutic effects on the target cells. As the MCS arrays are subjected to biological agents, the contraction of the underlying MCS-supporting micropattern can be ascertained based on the geometric size of the MCS-supporting micropattern, whereby a size smaller than that of the originally disposed micropattern size, prior to introduction of cells, indicates greater contraction.

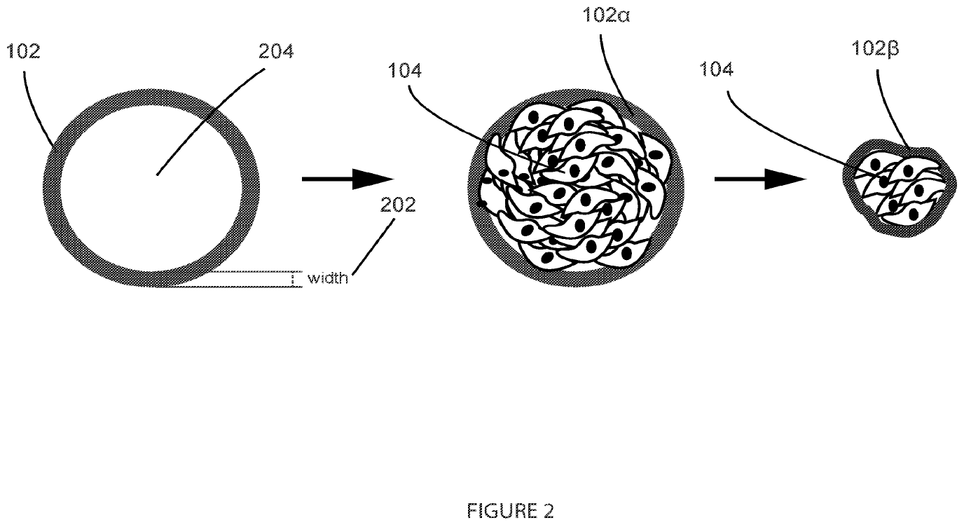

FIG. 2 illustrates one example of a MCS-supporting micropattern 102 that can be loaded with a plurality of target cells to form a MCS 104 for measuring cell forces, in accordance with some embodiments of the disclosure.

As depicted herein, a MCS-supporting micropattern 102 comprises a fluorophore-conjugated material with a particular width 206. In various embodiments, the width 206 of the fluorophore-conjugated material is between about 10 microns to about 100 microns. In various embodiments, the fluorophore-conjugated material exhibits cell adhesion properties or is co-patterned with a material that exhibits cell adhesion properties.

The MCS-supporting micropattern 102 also has a non-adhesive interior region 204 that does not contain fluorophore-conjugated or adhesive material. In addition to not possessing inherent adhesive properties, the unprinted interior region 204 of the MCS-supporting micropattern, as well as all non-patterned regions of the soft layer, are also chemically blocked from adsorption of cell-secreted proteins with a 30 minute exposure to 0.5% Pluronic F-127 solution. By preventing adsorption of cell-secreted proteins to the soft layer, the non-patterned regions, including the interior region 204 of the micropatterns remain non-adhesive to cells, indefinitely. In various embodiments, the area of the interior region is between about 7,500 square microns and about 200,000 square microns.

Once the MCS-supporting micropattern 102 has been deposited or embedded into the elastic substrate, target cells can be deposited proximate to the pattern 102 and incubated in conditions that promote the cells to divide and grow across the interior region 204 of the pattern 102 without creating adhesions to the substrate within the interior region. Because it is itself non-adhesive to cells the interior region 204 induces cells to form cell-cell adhesions and grow into three dimensions despite the cells only anchoring to and growing from a two-dimensional planar micropattern 102 surrounding the interior region 204. Importantly, this 3D-like growth of the MCS 104 that is supported by the exterior adhesive segments that lie along the convex envelope of the pattern 102 formed on a 2D planar surface is much easier and more robust to fabricate than a multi-layer 3D structure that supports MCS growth between anchoring regions such as in Legant et al. PNAS 2009. In addition, adhesion to a surrounding adhesive envelope instead of throughout the interior region 204 routes MCS generated forces to these local exterior segments which amplifies displacements needed to accurately measure the generated forces. FIG. 2 illustrates both the original shape of the MCS-supporting micropattern (102α) and the final shape of the MCS-supporting micropattern after being contracted by the adhered MCS (102β).

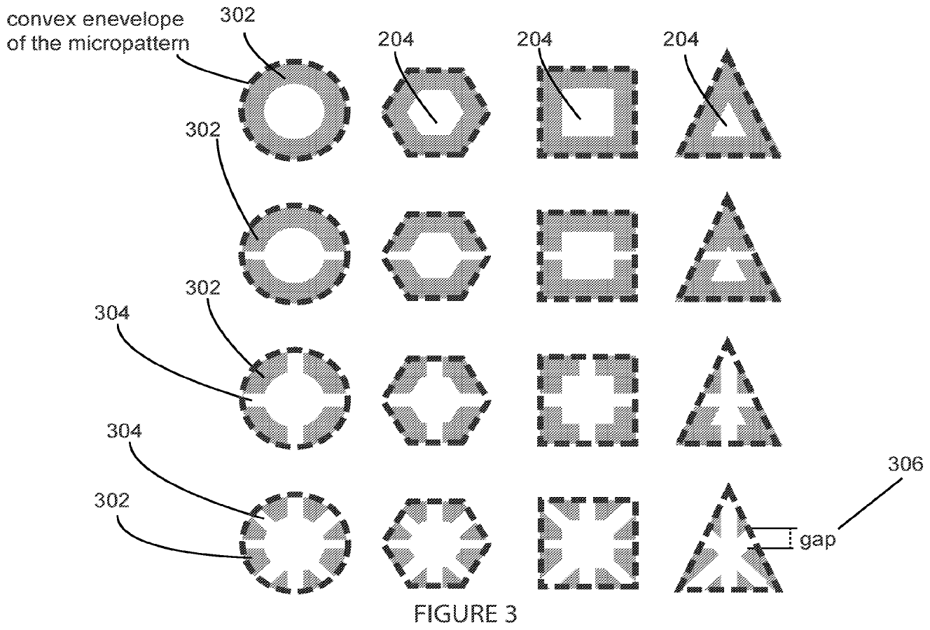

FIG. 3 illustrates illustrative shapes and configurations for the MCS-supporting micropatterns 102 that support MCS 104 growth and enable force generation measurements of the MCS, in accordance with some embodiments of the disclosure.

As depicted herein, within each of the plurality of MCS-supporting micropatterns 102 is an interior region 204 that is non-adhesive to cells. In various embodiments, the convex envelope of each MCS-supporting micropattern 102 possesses adhesive segments 302 that comprise adhesive and fluorescent materials, and the convex envelope of each MCS-supporting micropattern may optionally possess non-adhesive segments 304 that do not comprise adhesive and fluorescent materials and are non-adhesive to cells.

In order for the MCS 104 to span the entire non-adhesive interior region 204 without making adhesions to the interior region 204, it is important that adjacent adhesive segments 302 lying along the convex envelope, if the envelope comprises multiple adhesive segments, are in sufficiently close proximity to allow for cells adhering to one adhesive segment 302 to also adhere to any of the immediately adjacent adhesive segments 302. To ensure this is possible, the maximum linear distance or gap 306 between adjacent adhesive segments 302 along the envelope of the MCS-micropatterns 102 should not exceed about 50 micrometers.

In various embodiments, as depicted in FIG. 2, the curvature of the convex envelope of the MCS-supporting micropattern will guide cells to first adhere along the adhesive segments lying along the convex envelope. Then, cells may divide and adhere to other cells, which are adhered along the envelope, such that these cell-cell adhesions and growths begin to support cell adhesions across portions of the non-adhesive interior region 204 of the micropattern 102, following with its curvature, rather than out to adjacent micropatterns 102. Cells then continue to grow and adhere to adjacent cells that are already bridging between adhesive segments 302 across portions of the non-adhesive interior region 204 until a MCS 104 covers the entire of the non-adhesive interior region, still without making adhesions to this non-adhesive interior region.

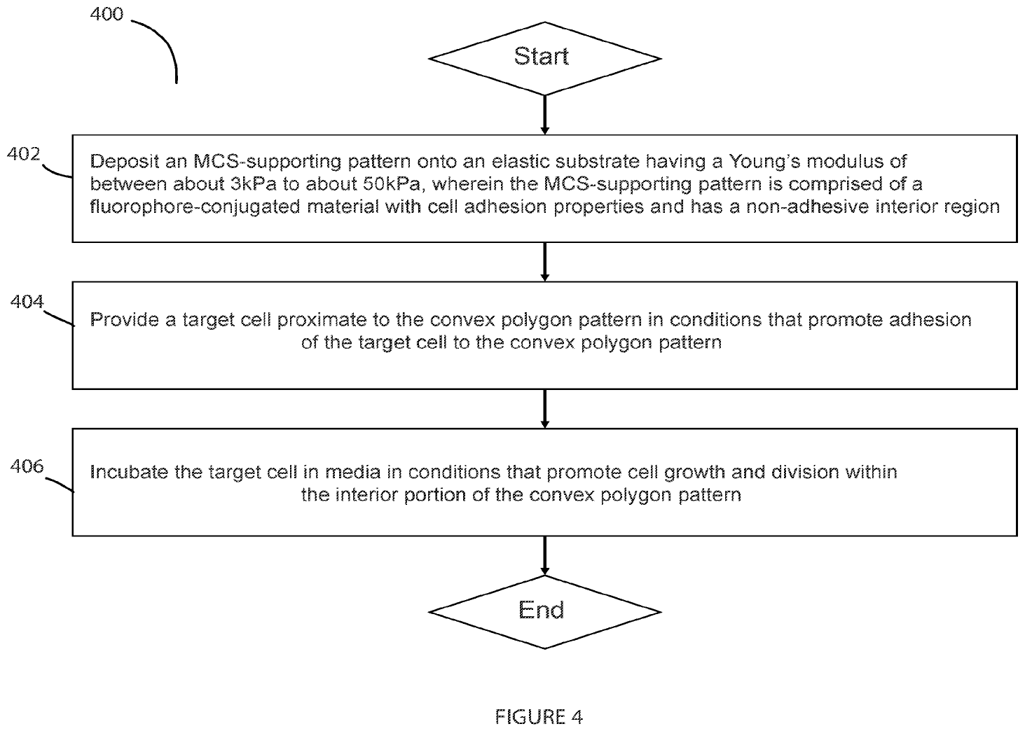

FIG. 4 is a flowchart showing a method 400 for producing an assay to measure cell-generated forces, in accordance with some embodiments of the disclosure.

As depicted herein, in step 402, a MCS-supporting micropattern 102 is deposited onto or embedded into a first substrate 106 having a Young's modulus of between about 0.1 kPa to about 50 kPa, wherein the MCS-supporting micropattern 102 is comprised of a fluorophore-conjugated material with cell adhesion properties 302.

In various embodiments, the first substrate 106 is an elastic material that is comprised of a silicone elastomer such as polydimethylsiloxane (PDMS).

In step 404, a plurality of target cells is provided proximate to the MCS-supporting micropattern 102 in conditions that promote adhesion of the target cell to the MCS-supporting micropattern 102. In one embodiment, these conditions include addition of Dulbecco's Modified Eagle's Medium supplemented with 10% fetal bovine serum and incubation at 37 degrees Celsius which enables both adhesion and division of cells.

In step 406, once the target cells are attached to the two-dimensional planar MCS-supporting micropattern 102, they are incubated at 37 degrees Celsius in media conditions that promote cell division and growth, and continue to grow and divide along the micropattern's adhesive segments lying on its convex envelope 302 and across the interior region 204 of the MCS-supporting micropattern 102, without binding to the substrate in the interior region 204. This cell division and growth combined with cell-cell adhesion creates a three-dimensional contiguous cellular structure 104 that is adhered to the adhesive segments lying on the micropattern's convex envelope and that extends above and across the non-adhesive interior region 204 MCS-supporting micropattern 102.

Since the interior region 204 is non-adhesive to cells due to absence of adhesive material and because of the Pluronic F-127 treatment, cells adhere only to the adhesive and fluorescent segments 302 lying on the micropattern's convex envelope, and to other cells. As cells adhere to and spread along the micropattern's adhesive segments, the closed curvature of the micropattern's adhesive segments will route cell growth along these exterior adhesive segments and to other cells also adhered to other adhesive segments 302, thus bridging the non-adhesive segments of the envelope 304 and the interior non-adhesive portion 204.

In various embodiments, a second substrate comprised of a rigid optically transparent material is attached to the first substrate, on the side that is opposite the side of the embedded MCS-supporting micropatterns 102. Examples, of rigid optically transparent materials can include, but not limited to: glass, rigid plastics, crystalline materials, etc. This material is naturally adhered to by soft silicone elastomers such as PDMS and provides a rigid support that enables handling of the substrate as well as maintains its planarity and prevents wrinkling, folding, or self-adhering of the soft film.

In various embodiments, a third substrate containing a plurality of apertures is adhered to the soft film on the side opposite the side that the second rigid transparent layer is adhered to soft film, such that when it is attached to the first substrate a plurality of wells is formed wherein each well contains one or more MCS-supporting micropatterns therein (e.g., a subset of patterns can be contained within each well). For example, the second substrate could include 96 wells as is used in a conventional 96 well test plate (of course other number of wells could be used).

In various embodiments, after the target cells are incubated to form a MCS 104 spanning the interior region of the pattern 204, the substrate is rinsed with an aqueous buffer solution to remove unattached cells, by manual or automated aspiration of the medium and subsequent replacement of the medium.

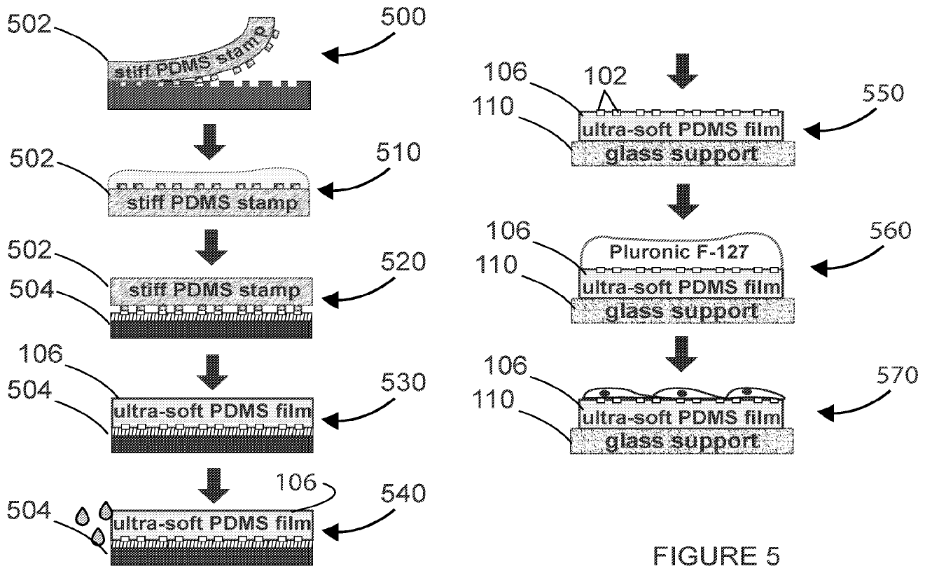

FIG. 5 depicts the explicit microfabrication process used to produce arrays of MCS-supporting patterns 100, in accordance with various embodiments. In step 500, a polydimethylsiloxane (PDMS)-based stamp 502 having a desired array of MCS-supporting micro-patterns is prepared. To form the stamp 502, a photoresist master mold is fabricated, using software such as “L-Edit” to design a metal photomask with the desired patterns. Fabrication of the chrome photomask is outsourced to one of a number of vendors known to one skilled in the art. A positive photoresist such as SPR-220-7 (thick resist) is spin-coated onto a silicon wafer, soft baked, exposed through the metal (e.g., chrome) photomask, and developed until the master mold is ready. The mold is taped to a petri dish and 10:1 ratio of base to crosslinker PDMS is poured over it and cured. The mold size is arbitrary and easily scalable.

In Step 510, once the stamp 502 is formed, the chosen adhesive molecule(s) are then inked onto the stamp by incubating the molecules with the stamp 502 for 5 or more minutes. For example, a protein solution is pipetted onto the stamp 502 and allowed to wet the entire surface.

In step 520, once the adhesive molecule(s) have adsorbed to stamp 502, the stamp is dried with pressurized air and pressed into contact with a dextran-coated silicon wafer 504. Dextran is purchased (Sigma-Aldrich) as a powder typically at the 70 kDa-100 kDa sizes. A 20% mass-by-volume solution is prepared in deionized water in a test tube. The tube is mixed continuously (taped to a vortex mixer for example) for up to 30 minutes or until the dextran is fully dissolved. Silicon wafers are treated with plasma for 30 seconds to improve their hydrophilicity and the dextran solution is spin-coated onto the wafer to form a uniform sub-micron height coating. After spin-coating, the dextran-coated wafers are baked at 150° C. for at least five (5) minutes to dry.

In step 530, after the stamping process uncured ultra-soft PDMS 106 is spin-coated onto the dextran coated wafer 504. The ultra-soft PDMS mixtures are prepared by mixing the base and curing agent in a sterile container and then placing into vacuum to remove air bubbles. Then, the PDMS is spin-coated on the dextran-coated wafers 504 that have been stamped with protein or another molecule to form the pattern. The target height is about 10-300 microns. After coating the wafers with the uncured soft-PDS mix, the gel is left to cure at 80 C.

In step 540, after curing, a second substrate, a transparent and rigid material such as a cover glass 110 is adhered to the exposed side of the soft PDMS film 106 by bringing the two faces into contact. Natural adhesive properties of the soft PDMS ensure a strong adhesion. Then, a sharp blade is used to cut and remove all the soft PDMS that is not covered by the rigid optically transparent material, from the dextran-coated wafer 504. Finally, this assembly is submerged in aqueous solution which dissolves the dextran layer.

In step 550, the glass-backed soft PDMS film with embedded MCS-supporting micropatterns 102 is released from the dextran-coated wafer 504 due to the dissolution of the dextran layer.

In step 560, the MCS-supporting micropattern array 100 is incubated in a 0.5% Pluronic F-127 solution for about 20 minutes to chemically blocked the non-patterned regions of the micropattern array 100 from cell adhesion and adsorption of cell-secreted proteins.

In step 570, target cells may be seeded on to the micropattern array 100 and incubated in the appropriate conditions to form contractile MCS.

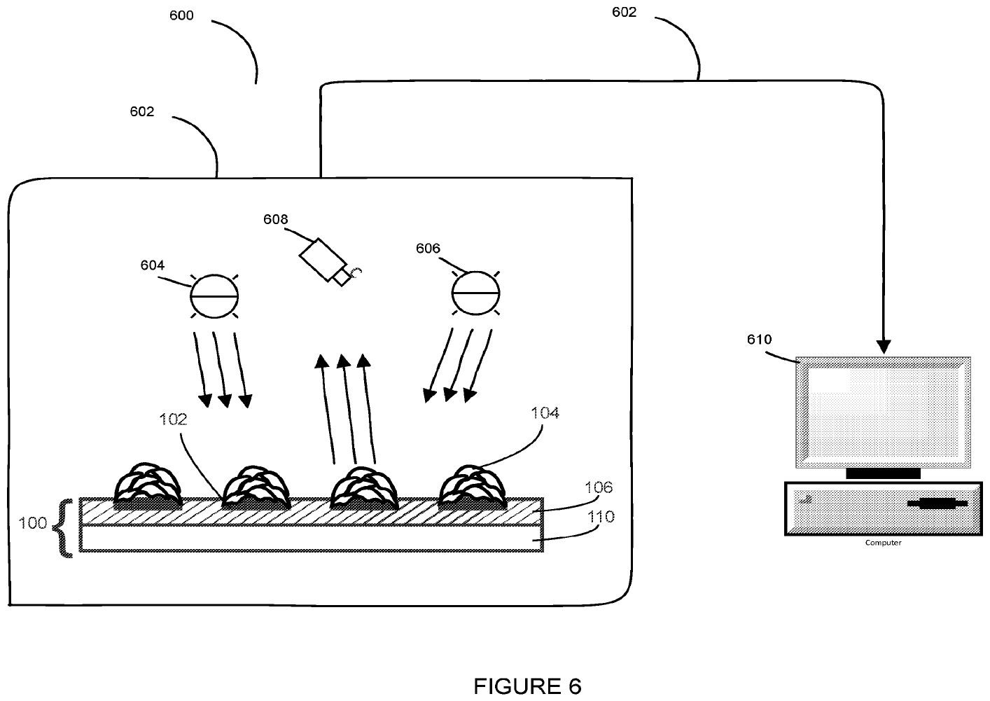

FIG. 6 is a schematic diagram of a system for measuring cell-generated forces, in accordance with various embodiments. As depicted herein, the system 600 includes an instrument housing 602 that houses a first light source 604, a detection module 608, an assay 600 and a computing device 610.

The assay 100 is further comprised of one or more MCS-supporting micropattern(s) 102 embedded into a top surface of a first substrate 106 that is itself attached to a second substrate 110 on bottom, and to a third layer comprising a plurality of apertures on the top. Each of the MCS-supporting micropatterns 102 is comprised of a fluorophore-conjugated material with cell adhesion properties, or of multiple materials of which at least one is fluorophore-conjugated and at least one has cell adhesion properties and has an interior region that is non-adhesive to cells 204. In various embodiments, the area of the interior region is between about 7,500 square microns and about 200,000 square microns. Within its boundaries including within the interior region of each MCS-supporting micropattern 102 is a MCS 104 comprised of a plurality of target cells which have been incubated so that they divide and grow in a controlled manner into a 3D contiguous multicellular structure 104 that spans the interior region of the MCS-supporting micropattern 102. After the MCS 104 is formed, it can be tested with various chemical or biological agents to characterize their respective toxicological and/or therapeutic effects on the target cells, such as the increase or decrease of the contraction of the multicellular structure, which is read out as a change in dimension of the MCS-supporting micropattern 102.

In various embodiments, the first substrate 106 is comprised of an elastic material having a Young's modulus of between about 0.1 kPa to about 50 kPa. In various embodiments, the first substrate is an elastic material that is comprised of silicone polymer such as polydimethylsiloxane (PDMS).

In various embodiments, the second substrate 110 is comprised of a rigid optically transparent material. Examples, of rigid optically transparent materials can include, but not limited to: glass, rigid plastics, crystalline materials, etc.

The first light source 604 is capable of emitting light at a first pre-determined wavelength to excite the fluorophore-conjugated material. For example, the first light source 404 can be a wavelength filtered arc lamp, a light emitting diode (LED) or laser diode or multiple LEDs or laser diodes. In various embodiments, the first light source 604 may be a tunable light source in which different wavelengths can be selected. That is, a single light source may be used instead of multiple light sources.

In various embodiments, the system 600 can also include a second light source 606 that is capable of emitting light at a different wavelength from the first light source 604. That is, the second light source 606 is capable of emitting light at a second predetermined wavelength to excite second fluorophore conjugate (attached to the target cells) that is different from the fluorophore-conjugated material.

The detection module 608 is operable to capture fluorescent light emitted by the MCS-supporting micropattern 102 and optionally the MCS 104. Examples of the types of detection modules that can be used include, but are not limited to: CCD, CMOS, etc. After capturing the fluorescent light emitted by the pattern 102 and/or MCS 104, detection module 408 generates imaging data and sends it to the computing device 610 by way of a hard-wire or wireless data connection 602.

The computing device 610 is capable of receiving the imaging data from the detection module 408 to measure dimensional changes in the MCS-supporting micropattern 102.

In various embodiments, the collected images are imported into an image processing software such as MATLAB or Python. The fluorescent images are thresholded to yield binary images. A characteristic dimension is computed for each micropattern, such as, for example, the total interior area, or the perimeter. The characteristic dimensions are compared to a reference, which is typically either the controlled and known original dimensions of the printed micropattern, or the original dimensions of that exact micropattern obtained at an earlier timepoint of interest. The smaller the dimension, the greater the contraction created by the MCS 104. The number of cells on each micropattern is also approximated by counting the number of imaged nuclei within the MCS 104, or by taking the total intensity of the signal in the channel in which the nuclei are labeled and comparing to the background signal in the same channel. The quantity of cells can be used to normalize the measurement obtained for the micropattern to enable fair comparison to other micropattern measurements taken of micropatterns that adhered a different number of cells.

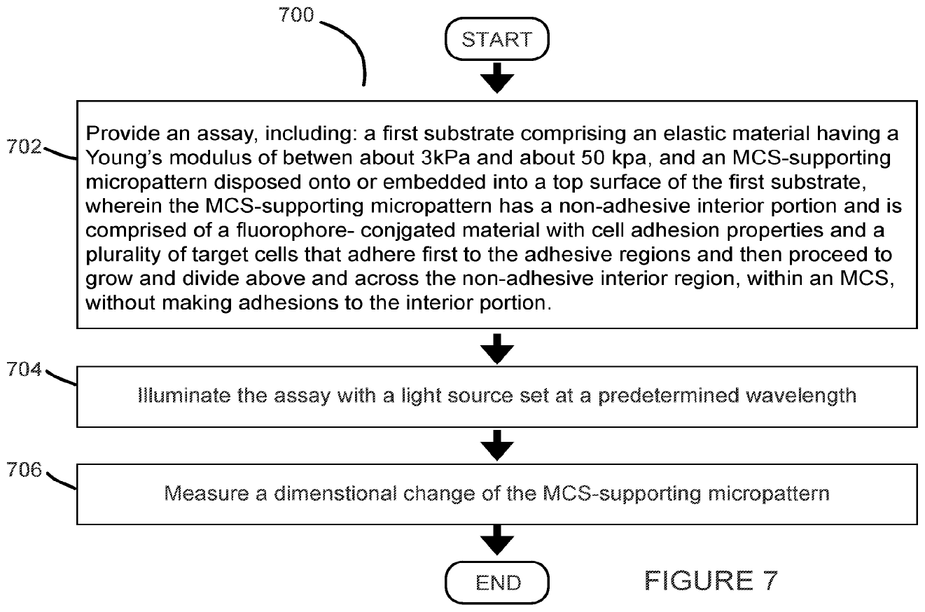

FIG. 7 is a flowchart showing a method for measuring cell-generated forces, in accordance with various embodiments. As depicted herein, method 700 details an exemplary workflow for measuring cell-generated forces on an assay. In step 702, an assay is provided. The assay includes a first substrate comprising an elastic material having a Young's modulus of between about 0.1 kPa to about 50 kPa and a MCS-supporting micropattern embedded into a top surface of the first substrate. The MCS-supporting micropattern is comprised of a fluorophore-conjugated material with cell adhesion properties, or of multiple materials of which at least one is fluorophore-conjugated, and at least one has cell adhesion properties, and a plurality of target cells confined within the boundaries of the MCS-supporting micropattern. The MCS-supporting micropattern also has an interior region that is non-adhesive to cells.

Examples of adhesive and fluorescent materials that can be used, include, but are not limited to fibronectin, fibrinogen, collagen type I, collagen type II, collagen type III, collagen type IV, gelatin, laminin, elastin, vitronectin, Matrigel, IgG, albumin, and poly-D-lysine, as well as other peptides and proteins. Any of these proteins may be conjugated to fluorescent moieties including red fluorescent protein (RFP), green fluorescent protein (GFP), Alexa Fluor 488, Alexa Fluor 555, Alexa Fluor 546, Alexa Fluor 568, Alex Fluor 647, Oregon Green, Fluorescein Isothiocyanate, tetramethyl rhodamine isothiocyanate, and other fluorescent molecules. In various embodiments, the conjugation chemistry between the fluorescent moieties and the adhesive molecules may involve amine-based and thiol-based conjugations.

In various embodiments, the elastic material is polydimethylsiloxane (PDMS).

In step 704, the assay is illuminated with a light source set a predetermined wavelength. The predetermined wavelength is selected to excite the fluorophore-conjugated material. The light source can be a wavelength filtered arc lamp, a light emitting diode (LED) or laser diode or multiple LEDs or laser diodes. In various embodiments, the light source may be a tunable light source in which different wavelengths can be selected. That is, a single light source may be used instead of multiple light sources.

In step 706, the dimensional change of the MCS-supporting micropattern is measured. This measurement is performed using image analysis software such as MATLAB or Python, in which the fluorescent image of the MCS-supporting micropatterns is first thresholded to yield a binary image, and each micropattern is detected using the regionprops or similar function for detecting binary objects. Then, each instance of a binary representation of the micropatterns is measured in terms of total interior area or perimeter, or other characteristic morphological measurement. In various embodiments, this characteristic measurement is performed for the same MCS-supporting micropattern that has been imaged multiple times, for example, at intervals ranging from milliseconds to days. In various embodiments, a test substance (chemical or biological) is introduced to the plurality of target cells prior to measuring the dimensional change of the MCS-supporting micropattern.

In various embodiments, the methods of the present teachings may be implemented as firmware and/or a software program and applications written in conventional programming languages such as C, C++, Python, etc.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

Utility in Various Biological Studies and Tests

Broadly speaking, the system and assay has applications in: (1) formation of a plurality of uniformly sized 3D multicellular structures (e.g. tissuoids), (2) functional collective cellular or tissuoid contractility evaluation (3) and drug discovery of compounds and other interventions affecting cell tissuoid contractility, The implementation described above, for example, is useful for measuring inherent tissue forces exerted through focal adhesions and provides a high-throughput method of first forming and then phenotyping a plurality of uniformly-sized and force-generating multi-cellular structures. Other implementations of this system provide ways of assaying various other biological behaviors and responses to stimuli as noted below.

Creation of Models of Drug-Resistant Diseased Tissues:

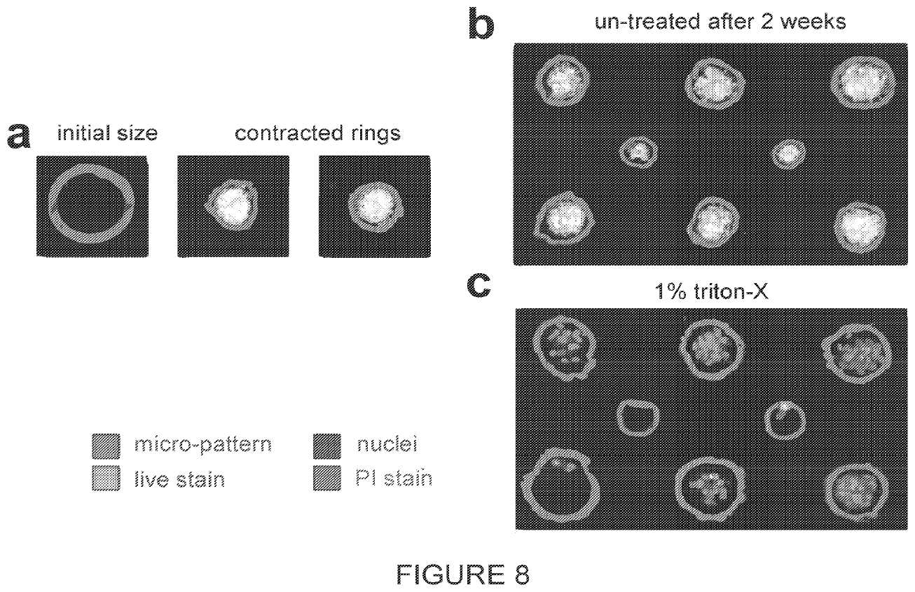

As shown in FIG. 8, MCS formed using the described system remain highly viable even following extensive time in culture, out to at least several weeks. This ability to maintain viable MCS while simultaneously performing phenotypic analyses, including measurements of contractility of the cells in the MCS, is unique to the MCS-supporting micropattern technique, and presents the opportunity to chronically subject the MCS to existing classes of drugs and induce resistance. Specifically, chronic receptor stimulation over the course of several weeks, which is not possible in single cells cultured on single-cell patterns because of their poor survivability beyond 24-48 hours, will result in down-regulation of those receptors in the cells comprising the MCS and create live models of treatment-resistant tissues. This presents opportunities to model treatment resistant contractility diseases where existing drugs are ineffective including treatment-resistant asthma, treatment resistant hypertension, and others.

Strain Due to Formation of Bio-Structures:

The system can be used to investigate and quantify the strain induced by the formation of various bio-structures such as multicellular bacterial biofilms as a function of structure size and duration of existence. Drugs could be screened that interfere with contractility of the biofilm or tissue-like structure (e.g., granuloma) which may be therapeutically useful to disrupt the biofilm or disaggregate the granuloma. Additionally, spores produced by certain bacteria have been shown to exert differential strain on substrates that depends on environmental conditions. See, e.g., Chen et al., O. Bacillus spores as building blocks for stimuli-responsive materials and nanogenerators. Nat Nano 9, 137-141 (2014). This system can be used to make simple and statistically significant comparisons of these responses against many environmental stimuli and assist in the development of spore-based stimuli-responsive materials.

Effects of Electrical Stimulation on Contractility:

This system would be especially useful for quantifying the contractile forces of excitable cells (e.g., neural cells, cardiomyocytes, smooth muscle cells) which are assembled to a more native multi-cellular or tissue arrangement in which they would survive longer than as single cells, as shown in FIG. 8.

Role of Genes in Contractility:

The system can be used in coordination with gene-silencing tools such as RNAi and CRISPR gene-editing technology to help identify which genes are most responsible for cell contractility and elucidate the pathways through which multiple genes work together to control contractility. Since cancer progression may rely on increased cell contractility, particularly for its role in migration and remodeling of the extracellular matrix (ECM), the system may be used to identify possible drug targets (e.g., proteins encoded by genes found to be implicated in contractility of malignant cells).

### EXAMPLE 1

### Experiments with Human Cells

FIG. 8 depicts a microscope image of MCSs 104 adhered to MCS-supporting micropatterns 102. FIG. 8a depicts the displacement and shrinking that occurs on micropatterns 102 that support MCS attachment and growth. When no MCS 104 is adhered to a micropattern 102, the geometry of the micropattern 102 conforms to the original geometry in which it was deposited onto or embedded into the first elastic layer 106. In various embodiments, when a MCS is adhered to a micropattern 102 then the micropattern is contracted and displaced under the stresses produced by the supported MCS 104.

Referring to FIG. 8b, MCS 104 supported by MCS-supporting micropatterns 102 are found to retain high viability even following extended culture times of several weeks. This provides a unique and considerable advantage over commonly used single-cell micropatterns that are designed to adhere exactly one cell and to limit its mobility, as cells in such isolation generally exhibit loss of viability after only a few days.

Referring now to FIG. 8c, analysis using live and dead staining in cells growing within MCSs adhering to MCS-supporting micropatterns 102 that are intentionally killed with treatment with 1% triton-X provide the negative control data to contrast the obvious high viability of cells growing within MCS 104 that have not been treated.

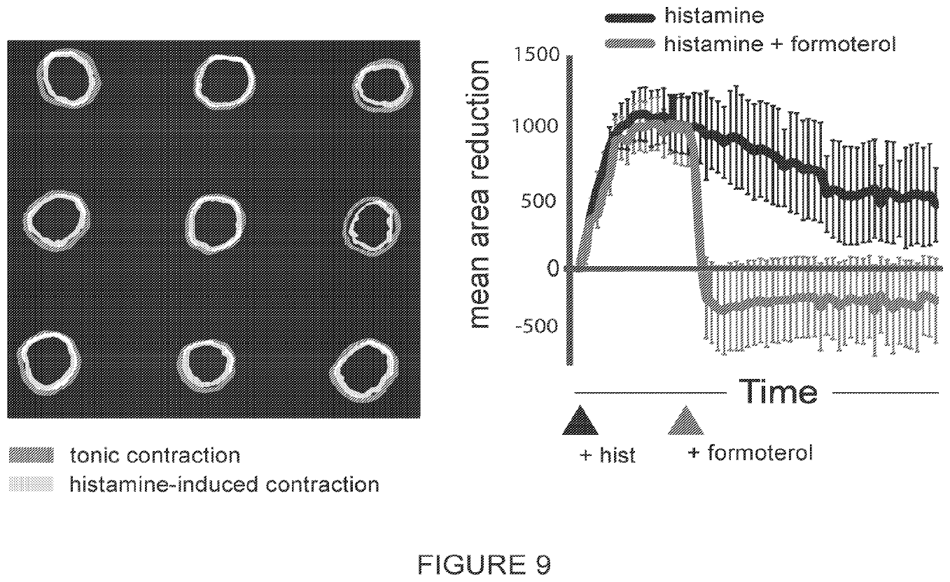

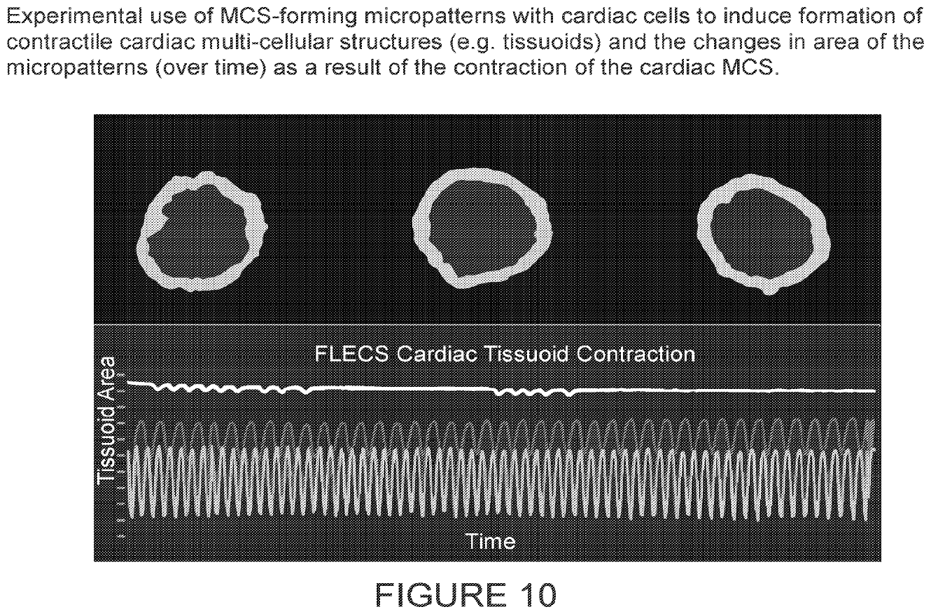

FIG. 9 depicts an overlay of two images obtained in the same field-of-view at different time points, containing an array of MCS-supporting micropatterns that have adhered fully formed MCSs comprising human airway smooth muscle cells and are contracted relative to their original size. Shown in this figure, at each position of this array are concentric MCS-supporting micropatterns, with the larger size representing the MCS baseline contracted state, and the smaller MCS-supporting micropatterns representing contraction of the very same MCS after the addition of histamine, a pro-contractile agonist. This figure also depicts a time course analysis a plurality of MCS-supporting micropatterns taken either from a single well on a 384-well plate that received only histamine, or from a different well on a 384-well plate that received first histamine and later formoterol. As clearly shown in the plot, the mean reduction in area of the MCS-supporting micropatterns taken from the second well, that also received formoterol, is significantly reduced, since the formoterol had a relaxing effect on the MCSs exposed to it, and the dimensions of the micropatterns adhering these MCSs in that well increased.

### EXAMPLE 2

### Exemplary Protocol to Generate MCS and Measuring their Contractility

To achieve the result depicted in FIG. 9, approximately 2000-2500 primary human airway smooth muscle cells were seeded into each of several wells on a microplate comprising 384 wells each with at least 60 MCS-supporting micropatterns deposed on their surface, in Ham's F12 medium supplemented with 10% serum and incubated for 5 days. The MCS-supporting micropatterns comprised a fluorophore-conjugated material comprising human fibrinogen conjugated to Alexa Fluor® 546 dye.

On day 5, MCS comprising the primary airway smooth muscle cells were observed within the wells. At this point, the serum-supplemented medium was replaced with serum-free medium to induce the contractile phenotype in the MCS. It is known to those skilled in the art that serum starvation of smooth muscle cells shifts the cells from a synthetic to a contractile phenotype. This step was taken to maximize contractile responsiveness to agonist in the next step.

After 24 hours of serum starvation, the microplate containing the MCS was imaged using a fluorescent microscope prior to any chemical addition in the fluorescent channel that corresponds to the fluorophore used in the MCS-supporting micropattern.

After imaging this initial timepoint, histamine was added to each well containing MCS to achieve a final concentration of 100 micromolar.

The wells containing the MCS treated with histamine were then again imaged for multiple timepoints at 1 minute intervals in the fluorescent channel that corresponds to the fluorophore used in the MCS-supporting micropattern.

After approximately 20 minutes of imaging, formoterol was added to half of the wells with MCS that previously received histamine. The other half of the wells received DMSO dissolved in medium at a concentration of DMSO equal that the DMSO concentration in the wells treated with formoterol.

Each well containing MCS that had been treated first with histamine and second with formoterol or with DMSO were imaged again for an additional 40 minutes at 1 minute intervals.

The images acquired were then analyzed using a MATLAB script written by the inventors that evaluated the initial areas of the MCS-supporting micropatterns, then tracked their change with time and drug addition, and finally averaged the result over each MCS-supporting micropattern that was treated with the same condition.

### EXAMPLE 3

### Experiments with Cardiac Myocytes

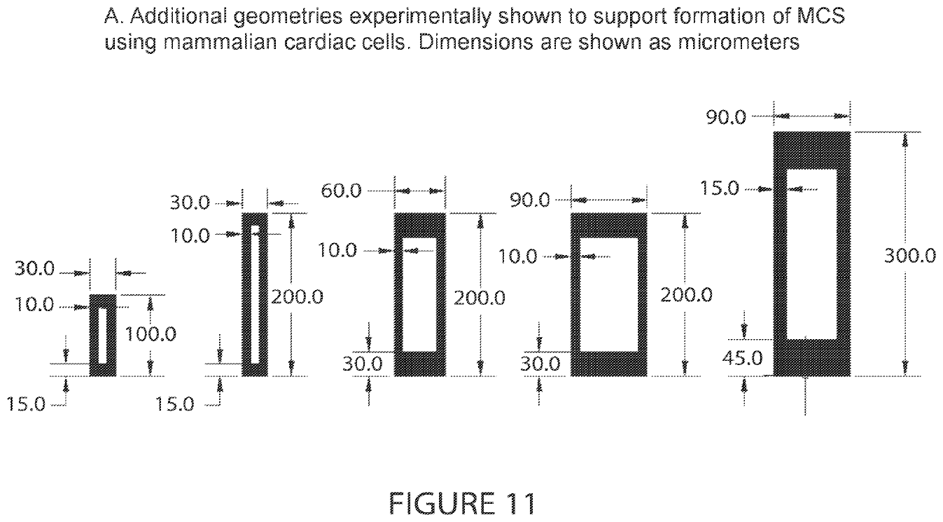

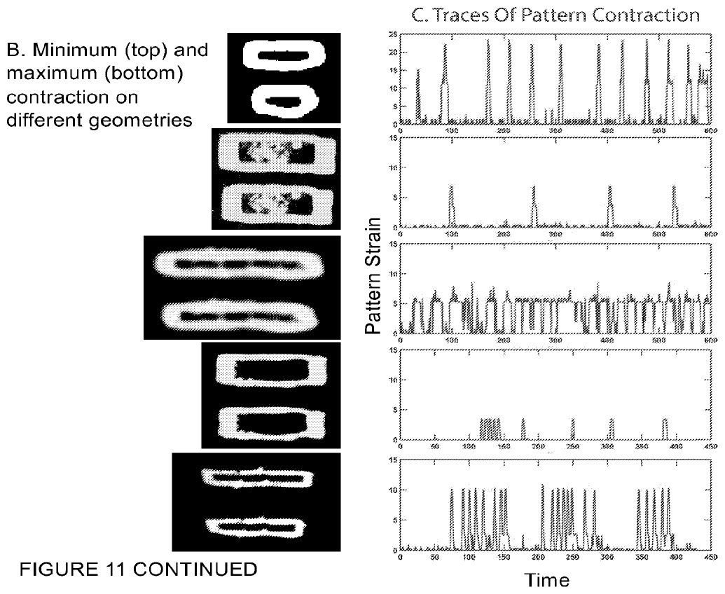

FIGS. 10 and 11b depict a microscope image of MCSs comprising cardiac myocytes 104 adhered to MCS-supporting micropatterns 102 of various forms. FIGS. 10 and 11c depicts the displacement and shrinking that occurs on micropatterns 102 that support MCS attachment and contraction. The MCS-supporting micropatterns comprised a fluorophore-conjugated material comprising human fibrinogen conjugated to Alexa Fluor® 546 dye. Since cardiac myocytes have distinct phasic beating patterns, it is observed that the areas of the MCS-supporting micropatterns transition from smaller to larger at particular interval on the order of seconds, corresponding to the beating of the adhered cardiac MCS.

What is claimed:

1. An assay for measuring cell-generated forces, comprising:
a first substrate comprising an elastic material having a Young's modulus of between about 0.1 kPa to about 50 kPa; and
a geometric micropattern disposed onto a surface of the substrate, wherein the micropattern comprises at least one segment consisting of a fluorophore-conjugated material and a material with cell adhesion properties, and the micropattern has an interior region.

2. The assay of claim 1, wherein the micropattern consists of a single material that is both fluorophore-conjugated and has cell adhesion properties

3. The assay of claim 1, wherein the interior region of the micropattern is non-adhesive to cells

4. The assay of claim 1, wherein the area of the interior region of the micropattern is between about 7,500 square microns and about 200,000 square microns.

5. The assay of claim 1, wherein a width of the fluorophore-conjugated material that forms the boundaries of the micropattern is between about 5 microns to about 100 microns.

6. The assay of claim 1, wherein the first substrate is further disposed onto a second substrate comprised of a rigid optically transparent material

7. The assay of claim 1, wherein first substrate comprises polydimethylsiloxane (PDMS).

8. An array for measuring cell-generated forces, comprising:
a first substrate comprising an elastic material having a Young's modulus of between about 0.1 kPa to about 50 kPa; and
a plurality of geometric micropatterns disposed onto a surface of the substrate, wherein each of the plurality of micropatterns is comprised of at least one segment consisting of a fluorophore-conjugated material and a material with cell adhesion properties, and the micropattern has an interior region.

9. The array of claim 8, wherein each of the plurality of micropatterns consists of a single material that is both fluorophore-conjugated and has cell adhesion properties

10. The array of claim 8, wherein the interior region is non-adhesive to cells

11. The array of claim 8, wherein an internal surface area of the interior region of the micropattern is between about 7,500 square microns and about 200,000 square microns.

12. The array of claim 8, wherein a width of the fluorophore-conjugated material that forms the boundaries of each of the plurality of micropatterns is between about 5 microns to about 100 microns.

13. The array of claim 8, wherein the first substrate is further disposed onto a second substrate comprised of a rigid optically transparent material

14. The array of claim 8, wherein first substrate comprises polydimethylsiloxane (PDMS).

15. A method for producing an assay to measure cell-generated forces, comprising:
depositing a geometric micropattern onto a planar first substrate having a Young's modulus of between about 0.1 kPa to about 50 kPa, wherein the micropattern comprises at least one segment consisting of a fluorophore-conjugated material and a material with cell adhesion properties, and the micropattern has an interior region.
providing a plurality of target cells proximate to the micropattern in conditions that promote adhesion of the target cells to the micropattern; and
incubating the target cells in media in conditions that promote cell growth and within the interior region.

16. The method of claim 15, further including:
attaching a second substrate comprised of a rigid optically transparent material to the first substrate.

17. A system for measuring cell-generated forces, comprising:
an assay, comprising:
a planar substrate comprising an elastic material having a Young's modulus of between about 0.1 kPa to about 50 kPa, and
a geometric micropattern disposed on a top surface of the substrate, wherein the micropattern comprises at least one segment consisting of a fluorophore-conjugated material and a material with cell adhesion properties, and the micropattern has an interior region;
a first light source capable of emitting light at a first pre-determined wavelength to excite the fluorophore-conjugated material;
a detection module operable to capture fluorescent light emitted by the micropattern; and
a computing device capable of receiving image data from the detection module to measure dimensional changes in the micropattern.

18. The system of claim 17, wherein the micropattern consists of a single material that is both fluorophore-conjugated and has cell adhesion properties

19. The system of claim 17, wherein the interior region of the micropattern is non-adhesive to cells

20. The system of claim 17, wherein the area of the interior region of the micropattern is between about 7,500 square microns and about 200,000 square microns.

21. The system of claim 17, wherein a width of the fluorophore-conjugated material that forms the boundaries of the micropattern is between about 5 microns to about 100 microns.

22. The system of claim 17, wherein the first substrate is further disposed onto a second substrate comprised of a rigid optically transparent material

23. The system of claim 17, wherein first substrate comprises polydimethylsiloxane (PDMS).

24. The system of claim 21, further including a second source capable of emitting light at a second predetermined wavelength to excite a second fluorophore conjugate attached to target cells within the boundaries of the micropattern.

25. The system of claim 21, wherein the detection module is further operable to capture fluorescent light emitted by the second fluorophore conjugates attached to the target cells.

26. The system of claim 21, wherein the micropattern consists of a single material that is both fluorophore-conjugated and has cell adhesion properties

27. A method for measuring cell-generated forces, comprising:
providing an assay, including:
a first planar substrate comprising an elastic material having a Young's modulus of between about 0.1 kPa to about 50 kPa, and
a geometric micropattern disposed onto a top surface of the first planar substrate, wherein the micropattern comprises at least one segment consisting of a fluorophore-conjugated material and a material with cell adhesion properties, and the micropattern has an interior region;
and a plurality of target cells confined within the boundaries of the micropattern;
illuminating the assay with a light source set at a predetermined wavelength; and
measuring a dimensional change of the micropattern.

28. The method of claim 27, further including:
introducing a test substance to the target cells prior to measuring the dimensional change of the micropattern.

29. The method of claim 27, further including:
measuring the dimensional change of the micropattern prior to introducing a test substance to the target cells, then introducing a test substance to the target cells, then taking another measurement of the dimensional change of the micropattern.
